(19) United States
(12) Patent Application Publication
(10) Pub. No.: US 2023/0079881 A1
(43) Pub. Date: Mar. 16, 2023

(54) DEVICES, SYSTEMS AND METHODS FOR SENSING AND DISCERNING BETWEEN FAT AND MUSCLE TISSUE DURING MEDICAL PROCEDURES

(71) Applicant: Apyx Medical Corporation, Clearwater, FL (US)

(72) Inventors: Shawn D. Roman, Safety Harbor, FL (US); Fredrik Jonsson, St. Petersburg, FL (US); Nikolay Dimitrov Shilev, Sofia (BG); Amin Elachchabi, Hollywood, FL (US); Gregory Goliszek, Oldsmar, FL (US); Viktor Tomov Tomov, Sofia (BG)

(21) Appl. No.: 17/799,469

(22) PCT Filed: Feb. 17, 2021
(86) PCT No.: PCT/US21/18281

§ 371 Date: Aug. 12, 2022

Related U.S. Application Data
(60) Provisional application No. 62/978,225, filed on Feb. 18, 2020.

(51) Int. Cl. A61M 1/00; A61B 18/04; A61B 18/14

(52) U.S. Cl. CPC A61M 1/895; A61B 18/042; A61B 18/1477; A61B 2017/00747

(57) ABSTRACT

The present disclosure is directed to devices, systems, and methods for sensing and discerning whether a distal portion of a fat grafting cannula or probe is disposed in fat tissue or muscle tissue during fat grafting procedures. In one aspect of the present disclosure, a fat grafting cannula or probe is provided including first and second electrodes. Each electrode is coupled to a circuit, e.g., disposed in an electrosurgical generator. During a fat grafting procedure, the electrodes contact patient tissue. Based on signals received from each electrode, the circuit is configured to determine whether a distal portion of the fat grafting cannula is disposed in a fat tissue or muscle tissue. If the circuit determines that the fat grafting cannula is disposed in muscle tissue, the surgeon operating the fat grafting cannula is alerted to ensure that processed fat is not injected into the muscle tissue of the patient.

### PRIORITY

This application claims priority to U.S. Provisional Patent Application Ser. No. 62/978,225, filed Feb. 18, 2020, entitled “DEVICES, SYSTEMS AND METHODS FOR SENSING AND DISCERNING BETWEEN FAT AND MUSCLE TISSUE DURING MEDICAL PROCEDURES”, the contents of which are hereby incorporated by reference in its entirety.

### BACKGROUND

### Field

The present disclosure relates generally to fat grafting and cannulas, and more particularly, to devices, systems, and methods for sensing and discerning between fat and muscle tissue during medical procedures, such as, fat grafting.

### Description of the Related Art

The process of fat grafting liposuctioned fat tissue holds much promise in the area of cosmetic procedures. The grafting of lipoaspirated fat is increasingly being recognized as a method of restoring volume defects and of improving body contour abnormalities such as may be found in the cheeks, the breast or the buttocks. In addition, tissue carefully harvested by liposuction has been shown to be rich in stem cells capable of regenerating tissue and of improving a number of conditions related to scarring, radiation damage and even aging.

Generally, fat grafting procedures include inserting a liposuction cannula into a tissue layer including adipose tissue or fat. The liposuction cannula is coupled to a controllable pressure mechanism (e.g., a syringe, fluid pump, etc.) configured to extract or aspirate the fat from the area the liposuction cannula has been inserted to. The extracted fat is collected (e.g., in a bag coupled to the controllable pressure mechanism). The extracted fat is then processed via centrifuging, filtering, and/or other techniques) to produce fat tissue or grafts in a form suitable for injection or grafting. The processed fat grafts are then injected or grafted into a target fat tissue layer or area of the patient (e.g., the buttocks, breast, etc.) using a fat grafting cannula or probe.

While fat grafting holds much promise, it is currently not without risks. One risk, which has resulted in a number of deaths (an occurrent rate of 1 in 3200 cases), is when the distal portion of the fat grafting cannula is inserted into subcutaneous tissue further than the fat layer and into muscle and processed fat is injected into the muscle layer rather than the fat layer. During procedures, such as gluteal fat grafting, injecting fat into the large vessels in the gluteal muscles can cause a fat embolism, which may result in the death of the patient.

Therefore, currently, there is an unmet need in the field of fat grafting to provide surgeons with a way to ensure the fat grafting cannula they are using is not disposed or inserted into a muscle layer when they are injecting processed fat during fat grafting procedures.

### SUMMARY

The present disclosure relates to devices, systems, and methods for sensing and discerning whether a distal portion of a fat grafting cannula or probe is disposed in fat or muscle tissue during fat grafting procedures.

In one aspect of the present disclosure, a fat grafting cannula or probe is provided including first and second electrodes. Each electrode is coupled to a circuit, e.g., disposed in an electrosurgical generator. During a fat grafting procedure, the electrodes contact patient tissue. Based on signals received from each electrode, the circuit is configured to determine whether a distal portion of the fat grafting cannula is disposed in fat tissue or muscle tissue. If the circuit determines that the fat grafting cannula is disposed in muscle tissue, the surgeon operating the fat grafting cannula is alerted to ensure that processed fat is not injected into the muscle tissue of the patient. In this way, fat embolisms and other dangers associated with injecting processed fat into the muscle tissue of a patient are avoided.

According to one aspect of the present disclosure, a fat grafting probe is provided including a base having a proximal end and a distal end, the base including a first fluid channel extending between the proximal and distal ends, the proximal end including an opening configured to receive an output of a pressure control device; a shaft including a proximal end coupled to the distal end of the base and a distal end including at least one aperture, the shaft includes a hollow interior operating as a second fluid channel which is in fluid communication with the first fluid channel; at least two electrodes associated with the shaft and coupled to an impedance detection circuit; and the impedance detection circuit that determines the impedance between the at least two electrodes and generates an indication whether the distal end of the shaft is in fat tissue or muscle tissue.

In another aspect, the impedance detection circuit includes a transformer having primary windings and secondary windings, the at least two electrodes being coupled to the secondary windings; a voltage controlled alternating current source coupled to one leg of the primary windings; and at least one processor coupled to the one leg of the primary windings to sense voltage on the one leg and determine the impedance between the at least two electrodes based on the sensed voltage.

In a further aspect, the impedance detection circuit further includes an interface module that provides the indication whether the distal end of the shaft is in fat tissue or muscle tissue.

In one aspect, the interface module is disposed on the base.

In another aspect, the impedance detection circuit further includes a low pass filter disposed between the second windings and the at least two electrodes to suppress radio frequency noise.

In a further aspect, if the impedance detection circuit determines the impedance is below a first predetermined setpoint, the distal end of the shaft is disposed in muscle tissue.

In another aspect, if the impedance detection circuit determines the impedance is above a second predetermined setpoint, the distal end of the shaft is disposed in fat tissue.

In yet another aspect, the probe further includes a communication module coupled to the at least one processor, the communication module communicates the indication to at least one other device.

In one aspect, a first electrode of the at least two electrodes is disposed on the distal end of the shaft and a second electrode is a return pad electrode.

In another aspect, the shaft is configured from a conductive material with an insulative sheath covering at least a portion of the shaft, an exposed portion of the shaft forming the first electrode.

In still another aspect, the shaft is configured from a conductive material with an insulative sheath covering at least a portion of the shaft, an exposed portion of the shaft forming a first electrode and a second electrode disposed on the sheath.

In a further aspect, the at least two electrodes are disposed at selected positions on the shaft, a first electrode being disposed a predetermined distance from a second electrode.

In one aspect, the probe further includes a connector for coupling conductive wire of the at least two electrodes to a power source, the connector including at least one memory configured to store parameters associated with the probe.

In another aspect, the probe further includes a connector for coupling conductive wire of the at least two electrodes to a power source, wherein at least a portion of the impedance detection circuit is disposed in the connector.

In a further aspect, the at least two electrodes are disposed on a connector that is removably coupled to the shaft.

According to another aspect of the present disclosure, a fat grafting system is provided including an electrosurgical generator configured for providing power; a probe coupled to the electrosurgical generator including: a base having a proximal end and a distal end, the base including a first fluid channel extending between the proximal and distal ends, the proximal end including an opening configured to receive an output of a pressure control device; a shaft including a proximal end coupled to the distal end of the base and a distal end including at least one aperture, the shaft includes a hollow interior operating as a second fluid channel which is in fluid communication with the first fluid channel; and at least two electrodes associated with the shaft and coupled to an impedance detection circuit; and the impedance detection circuit that determines the impedance between the at least two electrodes and generates an indication whether the distal end of the shaft is in fat tissue or muscle tissue.

In one aspect, the impedance detection circuit is disposed in the generator.

In another aspect, pressure control device provides processed fat to a layer of fat tissue of a patient via the first fluid channel, the second fluid channel and the at least one aperture. The pressure control device may be at least one of a syringe and/or a pump.

In one aspect, a display module is coupled to the interface module configured to display the indication, the display module disposed on a surface of a housing of the electrosurgical generator.

In a further aspect, the electrosurgical generator is further configured to be coupled to a plasma generator and provide an electrosurgical radio frequency signal to the plasma generator.

In one aspect, a frequency of an output of the voltage controlled alternating current source is selected to be different than a frequency of the electrosurgical radio frequency signal.

In a further aspect, the impedance detection circuit further comprising a communication module coupled to the at least one processor, the communication module communicates a control signal to the pressure control device when the at least one processor determines that the distal end of the shaft is in muscle tissue.

In yet another aspect, the probe further includes a connector for coupling conductive wire of the at least two electrodes to the electrosurgical generator, the connector including at least one memory configured to store parameters associated with the probe and transmit the parameters to at least one processor of the electrosurgical generator.

According to another aspect of the present disclosure, a fat grafting probe is provided including a base having a proximal end and a distal end, the base including a first fluid channel extending between the proximal and distal ends, the proximal end including an opening configured to receive an output of a pressure control device; a shaft including a proximal end coupled to the distal end of the base and a distal end including at least one aperture, the shaft includes a hollow interior operating as a second fluid channel which is in fluid communication with the first fluid channel; at least two sensors associated with the shaft and coupled to a detection circuit; and the detection circuit that determines whether the distal end of the shaft is in fat tissue or muscle tissue based on sensed parameters of the at least two sensors.

In one aspect, the at least two sensors include an acoustic emitter and an acoustic receiver, the acoustic emitter disposed a predetermined distance from the acoustic receiver, the detection circuit including at least one processor configured to determine attenuation of a signal emitted by the acoustic emitter and determines whether the distal end of the shaft is in fat tissue or muscle tissue based on the attenuated signal.

In a further aspect, the signal emitted by the acoustic emitter has at least one of a predetermined frequency and/or a predetermined amplitude.

In yet another aspect, at least two sensors include an acoustic emitter and an acoustic receiver, the acoustic emitter disposed a predetermined distance from the acoustic receiver, the detection circuit including at least one processor configured to determine a time of flight of a signal emitted by the acoustic emitter to the acoustic receiver and determines whether the distal end of the shaft is in fat tissue or muscle tissue based on the speed of the signal.

In a further aspect, the at least two sensors include a heating element and a temperature sensor, the heating element disposed a predetermined distance from the heat sensor, the detection circuit including at least one processor configured to determine heat capacity of tissue between the heating element and heat sensor and determines whether the distal end of the shaft is in fat tissue or muscle tissue based on the determined heat capacity.

In one aspect, the at least one processor determines the heat capacity by measuring a temperature difference sensed by the temperature sensor before and after a predetermined heat pulse is emitted by the heating element.

According to another aspect of the present disclosure, a method for performing a medical procedure includes inserting a distal end of a fat grafting cannula into a subcutaneous tissue plane; monitoring at least one property of tissue disposed proximately to the distal end of the fat grafting cannula, determining, based on the monitored at least one property, if the distal end of the fat grafting cannula is disposed on in fat tissue or muscle tissue; and generating an indication of whether the distal end is in fat tissue or muscle tissue.

In one aspect, if the distal end of the fat grafting cannula is disposed in fat tissue, generating an alert to proceed to inject processed fat into the fat tissue.

In another aspect, if the distal end of the fat grafting cannula is disposed in fat tissue, transmitting a signal to a processed fat pressure controlling device to proceed to inject processed fat into the fat tissue via the fat grafting cannula.

In a further aspect, if the distal end of the fat grafting cannula is disposed in muscle tissue, transmitting a signal to the processed fat pressure controlling device to stop providing processed fat to the fat grafting cannula.

In another aspect, if the distal end of the fat grafting cannula is disposed in muscle tissue, generating an alert that the distal end of the fat grafting cannula is disposed in muscle tissue.

In a further aspect, the at least one property includes at least one of electrical impedance, acoustic impedance and/or heat capacity.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is an illustration of a fat grafting system in accordance with an embodiment of the present disclosure;

FIG. 1B is a front view of an electrosurgical generator of the fat grafting system of FIG. 1A in accordance with an embodiment of the present disclosure;

FIG. 2 is a block diagram of a circuit of an electrosurgical unit of the fat grafting system of FIG. 1A in accordance with an embodiment of the present disclosure;

FIG. 3 is an illustration of another fat grafting system in accordance with an embodiment of the present disclosure;

FIG. 4 is an illustration of another fat grafting system in accordance with an embodiment of the present disclosure;

FIG. 5 is an illustration of another fat grafting system in accordance with an embodiment of the present disclosure;

FIG. 6 is an illustration of another fat grafting system in accordance with an embodiment of the present disclosure;

FIG. 7 is an illustration of another fat grafting system in accordance with an embodiment of the present disclosure;

FIG. 8 is an illustration of another fat grafting system in accordance with an embodiment of the present disclosure;

FIG. 9 is an illustration of another fat grafting system in accordance with an embodiment of the present disclosure;

FIG. 10 is an illustration of another fat grafting system in accordance with an embodiment of the present disclosure;

FIG. 11 is an illustration of another fat grafting system in accordance with an embodiment of the present disclosure;

FIG. 12 is an illustration of a connector for a fat grafting cannula in accordance with an embodiment of the present disclosure;

FIG. 13 is an illustration of another fat grafting system in accordance with an embodiment of the present disclosure;

FIG. 14 is a flow chart of a method in accordance with an embodiment of the present disclosure; and

FIG. 15 is another flow chart of a method in accordance with an embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, apparatus, applicator, handpiece, forceps, probe, etc., which is closer to the user, while the term “distal” will refer to the end which is further from the user. Herein, the phrase “coupled” is defined to mean directly connected to or indirectly connected with through one or more intermediate components. Such intermediate components may include both hardware and software based components.

The present disclosure is directed to devices, systems, and methods for sensing and discerning whether a distal portion of a fat grafting cannula or probe is disposed in fat or muscle tissue during fat grafting procedures. In one embodiment of the present disclosure, a fat grafting cannula or probe is provided including first and second electrodes. Each electrode is coupled to a circuit, e.g., disposed in an electrosurgical generator. During a fat grafting procedure, the electrodes contact patient tissue. Based on signals received from each electrode, the circuit is configured to determine whether a distal portion of the fat grafting cannula is disposed in fat tissue or muscle tissue. If the circuit determines that the fat grafting cannula is disposed in muscle tissue, the surgeon operating the fat grafting cannula is alerted to ensure that processed fat is not injected into the muscle tissue of the patient. In this way, fat embolisms and other dangers associated with injecting processed fat into the muscle tissue of a patient are avoided.

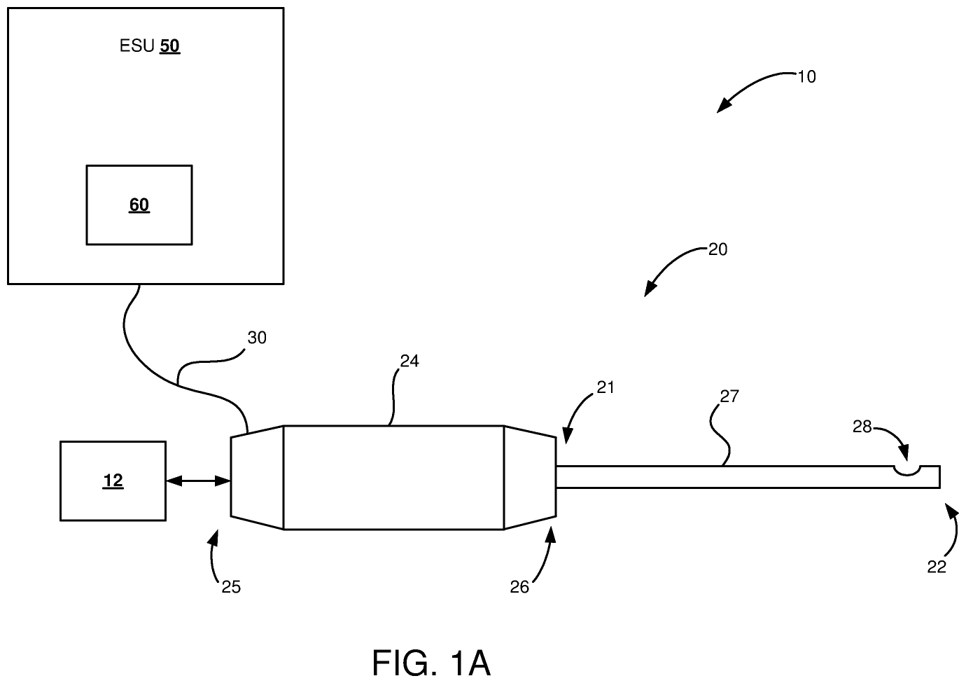

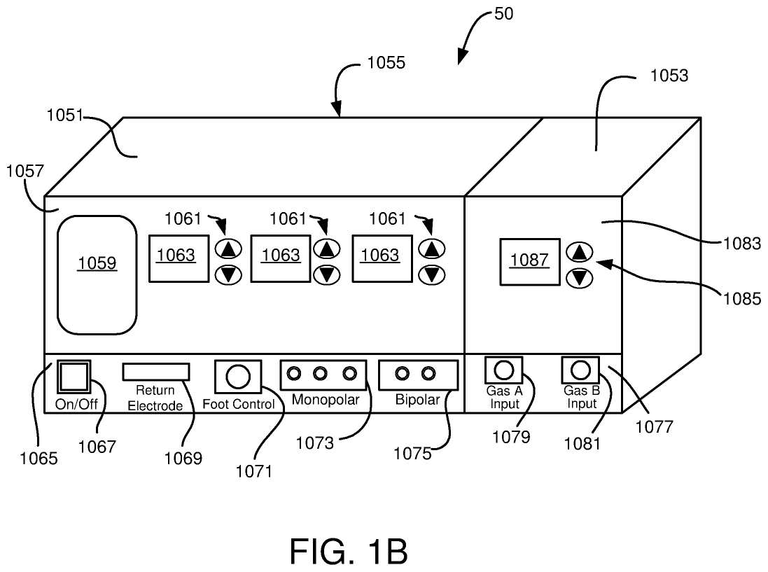

Referring to FIG. 1A, a fat grafting system 10 is shown in accordance with an embodiment of the present disclosure. System 10 includes an electrosurgical generator or energy source 50, a processed fat pressure controlling device 12, and a fat grafting cannula or probe 20.

Cannula 20 includes a hub or base 24 and a shaft or tubular portion 27, where shaft 27 includes a hollow interior operating as a fluid channel. Shaft 27 is coupled to and extends away from base 24 distally. Base 24 includes a proximal end 25 and a distal end 26. Shaft 27 includes a proximal end 21 (coupled to distal end 26 of base 24) and a distal end or tip 22. Distal end 22 includes one or more apertures 28. It is to be appreciated that, while FIG. 1A shows the aperture(s) 28 disposed at the distal end 22 of shaft 27, cannula 20 may be configured with any number of apertures disposed at various positions on shaft 27 and oriented in various directions.

The proximal end 25 of base 24 includes an opening (not shown) for receiving a pressure controlling device (e.g., a syringe, pump, or other pressure controlling device) 12, which proximal end 25 may be coupled to. The opening in proximal end 25 of base 24 reveals a fluid channel which is coupled to (i.e., in fluid communication with) the fluid channel within shaft 27. Prior to using fat grafting cannula 20, a liposuction cannula (not shown) is disposed or inserted into an adipose or fat tissue layer and the liposuction cannula is used to aspirate or extract fat from the adipose tissue. The extracted fat is collected into a collection device (e.g., a bag) coupled to liposuction cannula and processed to purify or refine the fat into grafts to be used in a fat grafting procedure. Thereafter, fat grafting cannula 20 is used to inject the grafts into a desired adipose tissue layer or plane. The distal portion of the shaft 27 is inserted into a desired adipose tissue layer or plane and the pressure controlling device 12 pumps the processed fat grafts through base 24, into shaft 27, and out of aperture 28 to be injected or grafted into the adipose tissue layer during a fat grafting procedure.

As shown in FIG. 1A, base 24 (e.g., via proximal end 25) optionally may be coupled to an electrosurgical generator or unit (ESU) 50 via a cable 30 including a plurality of conductors. In one embodiment, ESU 50 is configured for use with a plasma generating device or apparatus. ESU 50 is configured to provide electrosurgical energy and/or a gas to the plasma generating apparatus during electrosurgical procedures (e.g., skin tightening or other procedures). A front view of ESU 50 is shown in accordance with an embodiment of the present disclosure in FIG. 1B. In one embodiment, the ESU 50 may include a high frequency electrosurgical generator 1051 and gas flow controller 1053 contained in a single housing 1055. The ESU 50 may include a front panel face 1057 which includes an input/output section 1059, e.g. a touchscreen, for entering commands/data into the ESU 50 and for displaying data. The front panel 1057 may further include various level controls 1061 with corresponding indicators 1063. Additionally, the ESU 50 may include a receptacle section 1065 which may include an On/Off switch 1067, a return electrode receptacle 1069, a monopolar foot-switching receptacle 1071, monopolar hand-switching receptacle 1073 and a bipolar hand-switching receptacle 1075. The gas flow controller 1053 includes a gas receptacle portion 1077 which may further include a Gas A input receptacle 1079 and a Gas B input receptacle 1081. The gas flow controller 1053 may further include a user interface portion 1083 including selector switch or input 1085 and a display 1087. The selector switch or input 1085 enables selection of the type of gas being input, selection of a mixture of gases being input, a composition and/or percentages of a mixture of gases being input, a flow rate of a gas being applied to a handpiece or applicator, etc. It is to be appreciated that although FIG. 1B shows the high frequency electrosurgical generator 1061 and gas flow controller 1053 housed in a single housing 1055, gas flow controller 1053 may be provided as a separate, external device which interfaces with the ESU 50, via a wired and/or wireless interface.

In one embodiment, ESU 50 includes a circuit 60 for detecting tissue impedance. During a fat grafting procedure, circuit 60 may be used to determine when the distal end 22 of cannula 20 is disposed in a fat layer of tissue or a muscle layer of tissue. In this way, the dangers associated with injecting fat into a muscle layer may be avoided by using ESU 50 and cannula 20.

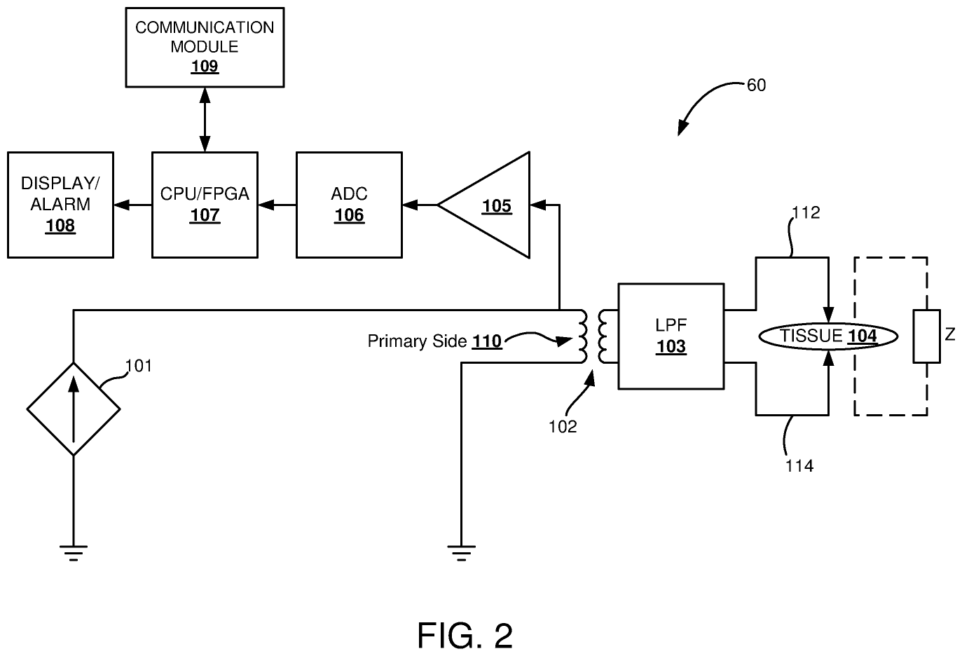

For example, referring to FIG. 2, circuit 60 is shown in accordance with an embodiment of the present disclosure. Circuit 60 includes a voltage controlled alternating current source 101, isolation transformer 102, low pass filter 103, amplifier or scaling component 105, analog to digital converter (ADC) 106, at least one processor (e.g., one or more CPUs and/or FPGAs) 107, and a display and alarm module 108. In circuit 60, current source 101 is coupled to the primary side winding 110 of transformer 102 and controls transformer 102 with a predetermined turn ratio (e.g., 1:1) between the primary side winding 110 and the secondary side winding of transformer 102. Primary side 110 of transformer 102 is further coupled to amplifier 105, which amplifies or scales the primary side voltage of transformer 102. The amplified voltage is converted from an analog signal to a digital stream by ADC 106 and then the digital stream is provided to processor 107. Based on the digital stream received by processor 107, processor 107 may display a value, activate one or more indicator lights, and/or activate an alarm using module 108. It is to be appreciated that module 108 may include one or more displays, indicator lights, and/or speakers controllable via processor 107. Any displays, lights, and speakers of module 108 may be disposed on a surface of the housing 1055 of ESU 50. Additionally, module 108 may transmit a value, indication and/or an alarm to be displayed on touchscreen 1059.

The secondary side winding of transformer 102 is, in some embodiments, coupled to a low pass filter (LPF) 103, which is configured to suppress certain types of radiofrequency (RF) noise. For example, LPF 103 may be used where ESU 50 is used as an electrosurgical generator with a plasma applicator. Where ESU 50 is used with a cannula 20, LPF may be bypassed (e.g., an alternative electrical path may couple transformer 102 to tissue 104) or removed. LPF 103 is coupled to leads or electrodes 112, 114, which are further coupled to patient tissue 104. As will be described in greater detail below, in some embodiments, a cannula 20 may include one or more electrodes, such as electrodes 112, 114 to be used with circuit 60.

When a current supplied by source 101 is provided across tissue 104 using electrodes 112, 114, the voltage on the primary side 110 of transformer 102 will be altered. The tissue 104 will include a tissue impedance Z that processor 107 is configured to determine based on the voltage on the primary side 110 of transformer 102. If the tissue impedance Z is high, the voltage across the primary side winding 110 of transformer 102 is also high and if the tissue impedance Z is low, the voltage is also low. In other words, the voltage on primary side 110 is associated with the tissue impedance Z. The primary side voltage is defined by the following equation, where Ivccs is the constant current provided from source 101:

Vpri=Ivccs×Z (1)

Using the digital streams (i.e., indicative of the voltage on primary side 110 of transformer 102), processor 107 is configured to determine the tissue impedance Z. Processor 107 may determine the tissue impedance using a lookup table stored in a memory coupled to processor 107, where the lookup table includes values relating the voltage sensed on primary side 110 of transformer 102, to impedance. It is to be appreciated that the lookup table values are calibrated for circuit 60 and for the cannula 20 being used. In some embodiments, processor 107 uses one or more equations, including equation (1) shown above, to determine impedance based on the voltage on the primary side 110 of transformer 102, as follows:

Z=Vpri/I vccs (2)

For example, the voltage and current on the primary side 110 of transformer 102 and the known turn ration between the primary side 110 and the secondary side of transformer 102 may be used to determine the voltage and current on the secondary side of transformer 102 and thereby the tissue impedance Z of tissue 104.

The tissue impedance is a non-linear function of the measured voltage on the primary side 110 of transformer 102. In general, the system 10 is calibrated at 8 different impedance values and the measured voltage readings are used in a cubic interpolation equation. The cubic interpolation is represented as lookup table coefficients, which are then used in the processor 107 calculation to define the value of the load impedance, based on the voltage reading at this impedance.

An exemplary lookup table is generated as follows. The equation for impedance Z between calibration values Ui and Uᵢ₊₁ is given by:

Z=Zi+A·(U−Ui)³+B·(U−Ui)²+C·(U−Ui)¹+D, (3)

where A, B, C, D are interpolation coefficients, Zi− impedance at Ui, U<Uᵢ₊₁
The first step in the process is to determine the interpolation coefficients (A, B, C, and D) of equation (3) above. For this process, approximately 30 corresponding pairs of voltage (U) and impedance (Z) are measured. The corresponding pairs of voltage (U) and impedance (Z) are established by applying a known impedance Z to the generator circuit 60 and measuring the corresponding voltage U value. This step generates 30 U values corresponding to 30 known Z values. With this many corresponding pairs of data, equation (3) can be solved for the interpolation coefficients (A, B, C, and D) to establish what A, B, C, and D will be going forward. For the purpose of illustrating the process of creating the look up table, voltage U and impedance Z pairs are provided in Table 1. It is to be appreciated that the values in Table 1 are not actual values but are merely for illustrative purposes.

TABLE 1
Example

| Known impedance | Corresponding Measured U |
|---|---|
| (ohms) | (volts) |
| Zᵢ = 10 | Uᵢ = 20 |
| Zᵢ₊₁ = 20 | Uᵢ₊₁ = 40 |
| Zᵢ₊₂ = 30 | Uᵢ₊₂ = 60 |
| Zᵢ₊₃ = 40 | Uᵢ₊₃ = 80 |
| Zᵢ₊₄ = 50 | Uᵢ₊₄ = 100 |
| Zᵢ₊₅ = 60 | Uᵢ₊₅ = 120 |
| Zᵢ₊₆ = 70 | Uᵢ₊₆ = 140 |
| Zᵢ₊₇ = 80 | Uᵢ₊₇ = 160 |
| and so on | |
| Zᵢ₊₃₀ = 310 | Uᵢ₊₃₀ = 620 |

Using the values from Table 1 above, 30 equations are generated. For example, Zᵢ₊₁=Zᵢ+A(Uᵢ₊₁−Uᵢ)3+B(Uᵢ₊₁−Uᵢ)2+C(Uᵢ₊₁−Uᵢ)1+D or using the numbers above

20=10+A(20)3+B(20)2+C(20)1+D

30=20+A(20)3+B(20)2+C(20)1+D

40=30+A(20)3+B(20)2+C(20)1+D

50=40+A(20)3+B(20)2+C(20)1+D

and so on. This results in 30 different equations such as above. Using these 30 equations, the interpolation coefficients A, B, C, and D can be solved. For the sake of this example, assume the interpolation coefficients are as follows: A=20.0, B=24.5, C=30.2, and D=15.6.

Next, the generator and/or circuit 60 is calibrated. At least eight 8 calibration points are used for each generator. For the sake of this example, assume the generator is calibrated using known impedances (Z) of 80 ohms, 180 ohms, 280 ohms, 380 ohms, 480 ohms, 580 ohms, 680 ohms, and 780 ohms. The voltage value U corresponding to each of the known impedances Z during calibration can then be measured. For example, the calibration results may produce pairs of known impedance Z to measured voltages U, as shown in Table 2 below.

TABLE 2
Calibration Results

| Known Z | Measured U |
|---|---|
| 80 | 20 |
| 180 | 21 |
| 280 | 22 |
| 380 | 23 |
| 480 | 24 |
| 580 | 25 |
| 680 | 26 |
| 780 | 27 |

With these calibration values, equation (3) above can be used with the established interpolation coefficients to calculate the impedances corresponding to voltages that occur that are between the calibration values shown in Table 2. Using the results of these calculations, look-up tables containing corresponding impedances for all voltages occurring between the calibration voltages in Table 2 can be established. In certain embodiments, a look-up table for a range of voltage values may be built. For example, a look-up table may be provided for voltages between 20 volts and 21 volts, one for between 21 volts and 22 volts, etc. The number of values in the look-up table is established based on the amount of resolution needed between values.

In use, the impedance is then determined based on the actual measurement of voltage on the primary side 110 of transformer 102. The actual voltage measurement is then used to find the impedance in the appropriate look up table. The determined impedance is then used to determine whether the distal end of the cannula is disposed in fat tissue or muscle tissue, as will be described in detail below.

If the impedance is determined to be below (or above) a predetermined threshold value by processor 107, processor 107 may display a notification via module 108 including a warning and the determined impedance and/or processor 107 may trigger an audible alarm and/or one or more indicator lights.

In some embodiments, processor 107 may determine one or more electrical properties based on the digital streams. For example, processor 107 may determine voltage and current. The processor 107 may then determine the phase shift between the voltage and current to determine the resistance R and reactance X of the impedance Z of the tissue 104. For example,

$$Z = \frac{{U}_{rms}}{{I}_{rms}} \cdot {e}^{i ∅} , R = \frac{{U}_{rms}}{{I}_{rms}} \cdot cos ∅ , X = \frac{{U}_{rms}}{{I}_{rms}} \cdot sin ∅ , \qquad ( 4 )$$

where Urms is the measured voltage, Irms is the measured current, i is an imaginary number, Ø—phase shift between voltage and current, R—resistance and X-reactance.
It is to be appreciated that the phase shift between voltage and current may be determined by the processor 107 by determining the zero crossings of each of the measured voltage waveform and measured current waveform and determining the difference. The determined impedance may then be used to determine the type of tissue the distal end 22 of shaft 27 is disposed in as will be described below.

In one embodiment, circuit 60 may be used for recognizing tissue impedance between a predetermined range, e.g., 10 ohms-2520 ohms. ESU 50 including circuit 60 may be used with cannula 20 during gluteal fat grafting to recognize whether the distal end 22 of shaft 27 is located in muscle tissue (e.g., less than 420 Ohm impedance) or in a fatty tissue (e.g., more than 1000 Ohm impedance). In one embodiment, processor 107 is configured to turn on a green indicator light of module 108 to indicate that the distal end 22 of shaft 27 of cannula 20 is disposed in adipose tissue. Processor 107 is further configured to turn on a red indicator light of module 108 and sound an alarm using a speaker of module 108 to indicate that the distal end 22 of shaft 27 of cannula 20 is disposed in muscular tissue. As will be described in greater detail below, in other embodiments, processor 107 may send one or more signals to cause indicator lights and/or or an alarm disposed in/on a cannula, such as cannula 20, to turn on or off to indicate whether distal end 22 of shaft 27 is disposed in fat tissue or muscle tissue.

In one embodiment, circuit 60 performs impedance monitoring with a low wattage (e.g., below 1 Watt) RF signal applied to the electrodes of cannula 20. In one embodiment, the peak values of the current associated with output of source 101 is selected so as not to exceed 10 mA. It is to be appreciated that the frequency of the output of source 101 is selected to be sufficiently different (e.g., not coincide with and be sufficiently far) from the frequencies of other alternating signals in generator 50 (e.g., for providing electrosurgical energy to a plasma generator or applicator), which may be generated by generator 50 concurrently with the output of source 101 for impedance monitoring described herein. For example, generator 50 may include the following signals and frequencies associated with the use of generator 50 in electrosurgery and/or plasma generating: carrier frequency (e.g., RF output of the generator 50 to be applied to patient using a plasma applicator), modulation frequency (e.g., a control signal for a power supply, such as for a switched-mode power supply), and a frequency associated with the recovery of the NEM (neutral electrode monitoring) signal. If the frequency of the output of source 101 is too close to (e.g., within a predetermined range of) the other signals produced by generator 50, a significant amount of noise may be produced and compromise the measurements obtained using circuit 60.

In some embodiments, the output of source 101 may be above 20 kHz to be sufficiently far from the neuromuscular stimulation. In some embodiments, the output of source 101 may be above 50 kHz to be sufficiently far from the modulation frequency, which may be in the range of 20 kHz up to 50 KHz. In some embodiment, the output of source 101 may be below 100 kHz to be sufficiently far from the highest noise introducer of the signals in generator 50—the RF output or carrier frequency. In one embodiment, the selected frequency of the output of source 101 is 100 kHz to be sufficiently far from the frequency associated with the recovery of the NEM (e.g., 62.5 kHz) and the RF output or carrier frequency (e.g., 100 kHz). In one embodiment, the selected frequency of the output of source 101 is substantially in the range of 80-100 kHz. It is to be appreciated that the above-described frequency values are merely exemplary and other values may be used without deviating from the scope of the present disclosure. In any case, the frequency of the output of source 101 is selected to be sufficiently spaced from all relevant frequencies generated by signals of generator 50 to preserve the accuracy of measurements of circuit 60 and reduce noise introduction.

In one embodiment, LPF 103 is configured to filter some of the noise introduced into circuit 60 by the frequencies of the signals of generator 50.

In one embodiment, processor 107 is communicatively coupled to pressure controlling device 12 via communication module 109, such that if processor 107 determines that the distal end 22 of shaft 27 is located in muscle tissue during a fat grafting procedure, processor 107 sends a single to pressure controlling device 12 to stop pumping or injecting processed fat into shaft 27 via base 24. In this way, processed fat is prevented from being injected into muscle tissue during the fat grafting procedure.

It is to be appreciated that the processor 107 and pressure controlling device 12 may be communicatively coupled via communication module 109 by hardwired and/or wireless connectivity. The hardwire connection may include but is not limited to hard wire cabling e.g., parallel or serial cables, RS232, RS485, USB cable, Firewire (1394 connectivity) cables, Ethernet, and the appropriate communication port configuration. The wireless connection may operate under any of the various wireless protocols including but not limited to Bluetooth™ interconnectivity, infrared connectivity, radio transmission connectivity including computer digital signal broadcasting and reception commonly referred to as Wi-Fi or 802.11.X (where x denotes the type of transmission), satellite transmission or any other type of communication protocols, communication architecture or systems currently existing or to be developed for wirelessly transmitting data including spread spectrum 900 MHz, or other frequencies, Zigbee, and/or any mesh enabled wireless communication.

As described below, cannula 20 may be configured with an active electrode and a return electrode, either in monopolar or bipolar arrangements, where circuit 60 may be used with either arrangement.

It is to be appreciated that various cannulas are described below in accordance with embodiments of the present disclosure. In each of the cannulas described below, unless otherwise indicated, components of each cannula that are similarly numbered to corresponding components of cannula 20 shown in FIG. 1A are configured in the manner and with the features described above and may not be described again below in the interest of brevity.

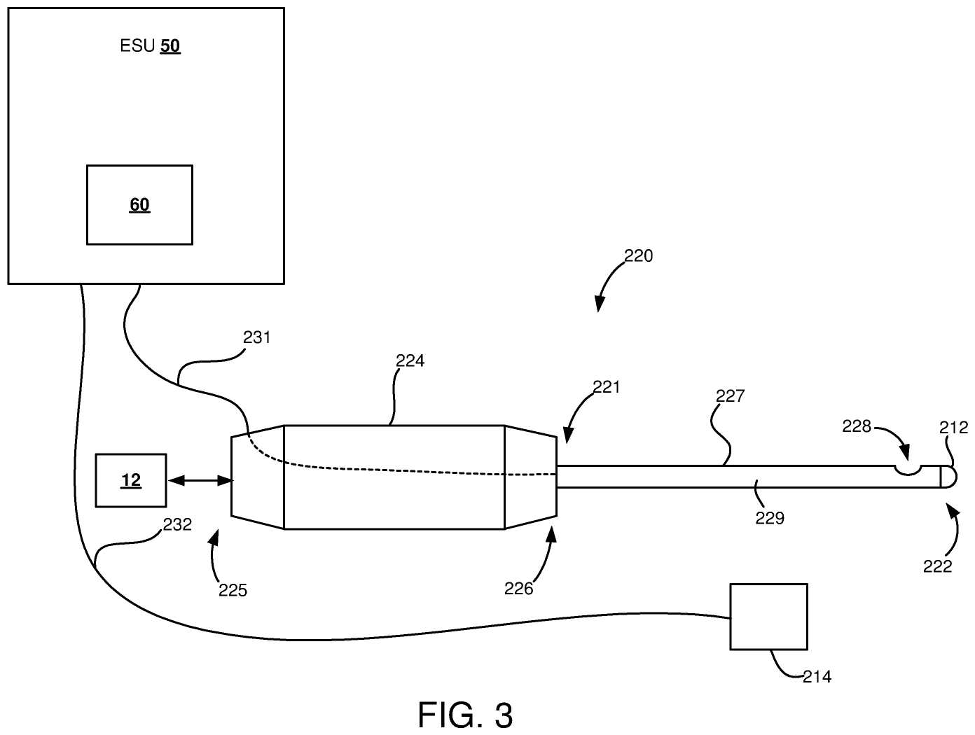

Referring to FIG. 3, a cannula 220 having a monopolar electrode arrangement is shown coupled to ESU 50 in accordance with an embodiment of the present disclosure. Cannula 220 includes a shaft 227 having a proximal end 221, a distal end 222, and an aperture 228. Shaft 227 is configured from a conductive material. An insulative sheath 229 is disposed over the exterior of shaft 227, such that a distal portion 212 of the conductive shaft 227 is exposed, forming a first, active electrode at end 222 of shaft 227. Within base 224, proximal end 221 of shaft 227 is coupled to an end of conductive wire 231, where an opposite end of conductive wire 231 is coupled to circuit 60 of ESU 50. The system shown in FIG. 3 further includes a grounding pad or return electrode 214, which is coupled to circuit 60 of ESU 50 via a conductive wire 232. For example, in one embodiment, electrode 212 is coupled to monopolar port 1073 via wire 231 and return electrode 214 is coupled to return electrode port 1069 via wire 232.

Electrodes 212, 214 are coupled to the secondary side winding of transformer 102, shown in FIG. 2, via wires 231, 232. Thus, when end 212 of cannula 220 is disposed in a subcutaneous tissue plane during a fat grafting procedure, a voltage signal received from circuit 60 may be applied across electrodes 212, 214 through the tissue plane that end 212 of cannula 220 is disposed in. As described above, processor 107 is configured to determine, based on the signals received from primary side winding 110 of transformer 102, the impedance of the patient tissue. Based on the determined impedance, processor 107 is configured to determine if end 212 of shaft 227 is disposed in fat tissue or muscle tissue, as described above.

It is to be appreciated that, while end 222 of shaft 207 is shown exposed to form electrode 212 in FIG. 2, in other embodiments of the present disclosure other portions of shaft 207 may be exposed instead of end 222 to alter the location of electrode 212.

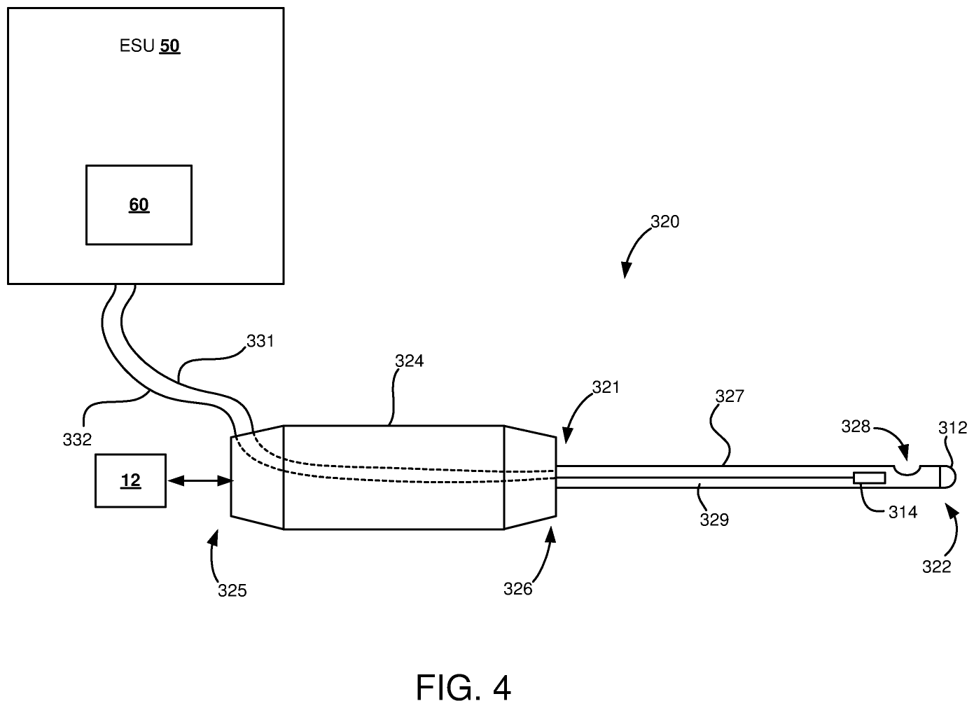

It is to be appreciated that cannula 220 may be adapted to a bipolar arrangement. For example, referring to FIG. 4, a cannula 320 configured in a bipolar arrangement for use with ESU 50 is shown in accordance with an embodiment of the present disclosure. Cannula 312 includes conductive shaft 327, which is covered by an insulating sheath 329 and includes an exposed tip 312, which forms an active electrode. A return electrode 314 is disposed over insulating sheath 329 (thus being electrically insulated from electrode 312) and coupled via conductive wire 332 to circuit 60. In one embodiment, wire 332 is disposed over sheath 329 and insulated. In another embodiment, wire 332 is embedded in sheath 329 without contacting shaft 327. In either case, electrode 314 forms a return electrode. Electrodes 312, 314 are coupled to the secondary side winding of transformer 102 of circuit 60, such that processor 107 can determine whether distal end 322 is disposed in fat tissue or muscle tissue using the determined impedance of tissue between electrodes 312, 322. In one embodiment, wires 331, 332 may be coupled to a connector configured to be mated with bipolar port 1075 of ESU 50 as shown in FIG. 1B.

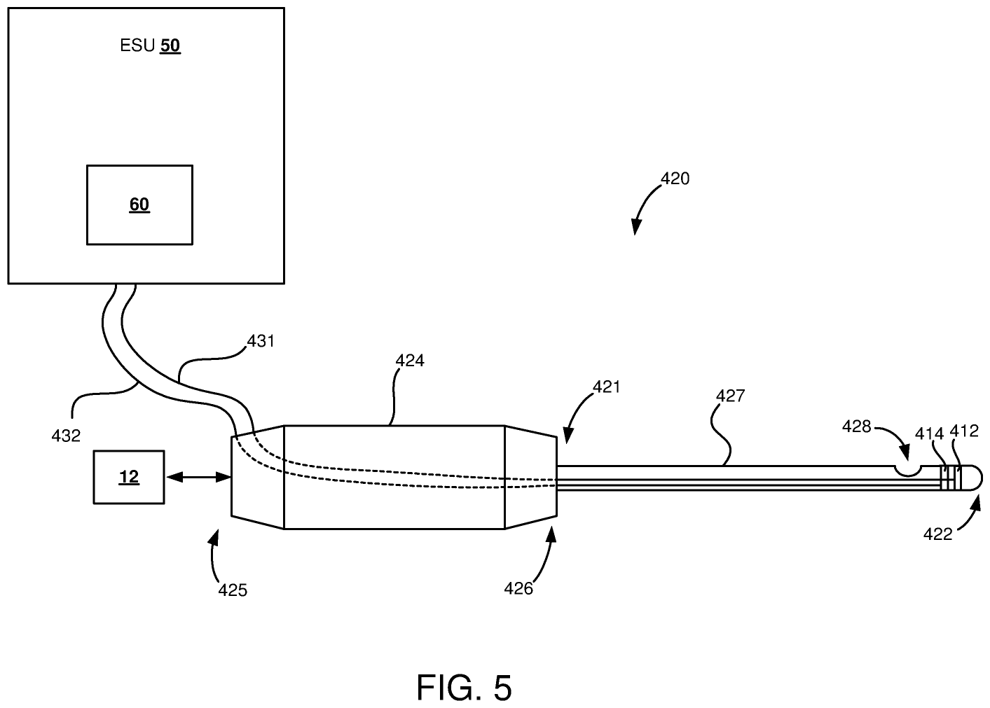

In one embodiment, shafts 227, 327 described above may be made of a nonconductive or insulative material and the active 212, 312 and return 214, 314 electrodes of each cannula 220, 320 may be disposed at selected positions of shafts 227, 327. For example, referring to FIG. 5, a bipolar configuration of a cannula 420 for use with ESU 50 including a nonconducting shaft 427 and electrodes 412, 414, is shown in accordance with the present disclosure. In this embodiment, active electrode 412 and return electrode 414 are disposed around the exterior of shaft 427 distally from aperture 428 with electrode 412 disposed distally with respect to electrode 414. Each of electrodes 412, 428 may be configured as a ring wrapping around the exterior of shaft 427. Electrode 412 is coupled via wire 431 to circuit 60 of ESU 50 and electrode 414 is coupled via wire 432 to circuit 60 of ESU 50. In some embodiments, wires 412, 414 may be disposed on the exterior of shaft 427. In other embodiments, wire 412, 414 may be embedded in a wall of shaft 427 or in the interior of shaft 427. Processor 107 of circuit 60 may determine if end 422 is disposed in fat tissue or muscle tissue in the manner described above.

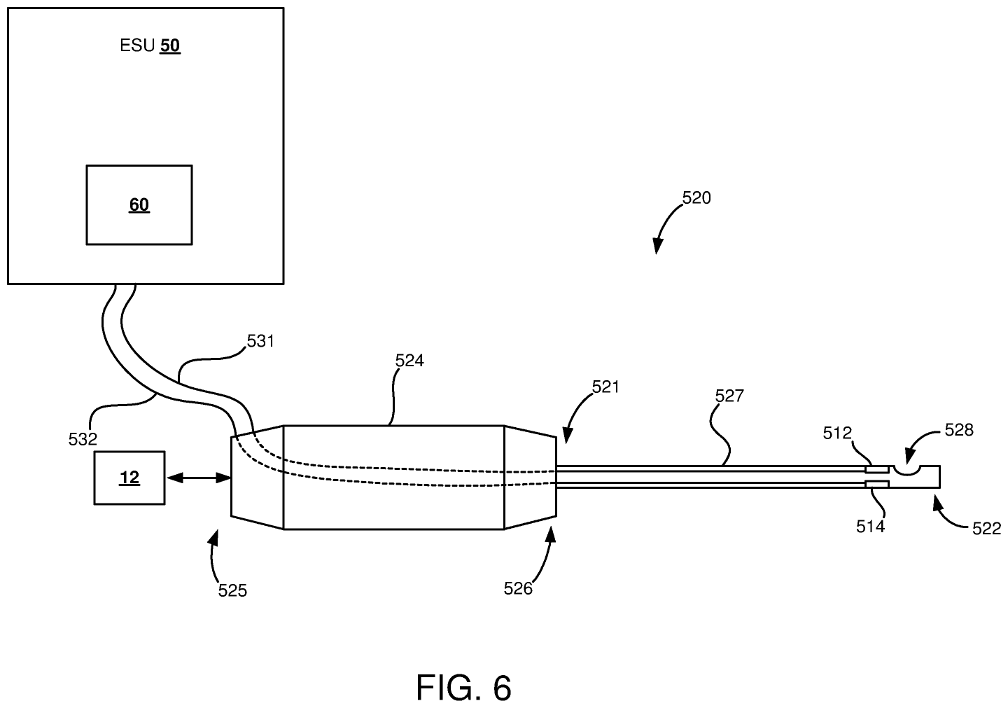

It is to be appreciated that electrodes 412, 414 may be disposed at various positions along shaft 427. For example, referring to FIG. 6, a cannula 520 for use with ESU 50 is shown in accordance with the present disclosure. Cannula 520 includes a shaft 527 that is nonconducting and electrodes 512, 514. Electrodes 512, 514 are disposed on the exterior of shaft 527 and are spaced apart about shaft 527 with electrodes 512, 514 disposed approximately the same distance from distal end 522. Electrode 512 is coupled to circuit 60 via conductive wire 531 and electrode 514 is coupled to circuit 60 via conductive wire 532. Processor 107 of circuit 60 may determine if end 522 is disposed in fat tissue or muscle tissue in the manner described above.

It is to be appreciated that any of the cannulas described above may include a connector for coupling the conductive wires and electrodes to ESU 50 and circuit 60. In one embodiment, the connector may include one-wire chip or a memory configured to store parameters associated with the cannula, where the memory may be read by processor 107 to enable ESU 50 enter into a first mode or a second mode of operation.

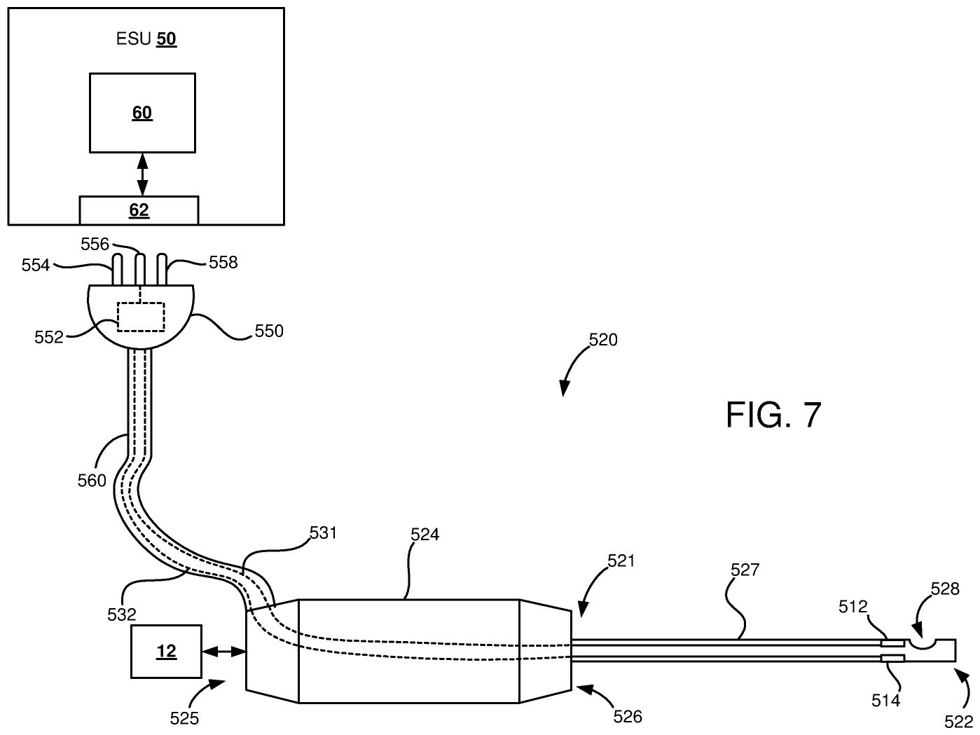

For example, referring to FIG. 7, cannula 520 is shown including a connector 550 and a cable 560. Cable 560 includes a portion of wires 531, 532 and couples connector 550 to base 524. Connector 550 include at least one memory or one-wire chip 552 configured to store information or parameters associated with cannula (e.g., the model or type of cannula and any other relevant parameters). Connector 550 is configured to be received by a port or receptacle 62 of ESU 50, for example, monopolar port 1073 or bipolar port 1075 as shown in FIG. 1B. Connector 550 includes at least 3 prongs, where, when connector 550 is coupled to receptacle 62 of ESU 50, a first prong 554 couples wire 532 to transformer 102 of circuit 60, a second prong 556 couples memory 552 to processor 107, and a third prong couples wire 521 to transformer 102 of circuit 60. Processor 107 is configured to read the parameters on memory 552 to cause ESU 50 to enter into a first mode, where ESU 50 uses circuit 60 to function as an impedance measuring device. When a plasma applicator is coupled to ESU 50, processor 107 does not detect memory 552, and thus ESU 50 enters a second mode, where ESU 50 functions as an electrosurgical generator for providing electrosurgical energy (and, in some embodiments, inert gas) to the plasma applicator.

It is to be appreciated that various types of information may be stored in memory 552 that may be read by processor 107. For example, memory 552 may include information associated with the energy to be applied across electrodes 512, 514 and/or other calibration information that enables ESU 50 and circuit 60 to function properly and make accurate determinations when used with cannula 520. In other embodiments, the memory 552 may have read/write capabilities, where the memory 552 may store how many times the cannula has been used and provide that information to processor 107.

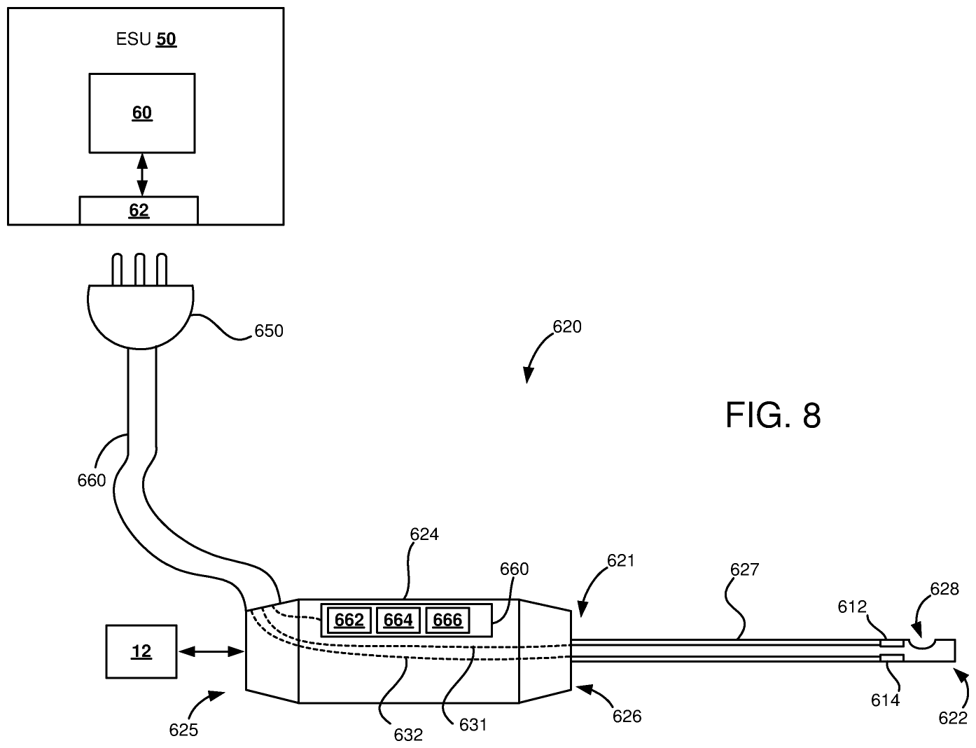

It is to be appreciated that any of the cannulas described above may be configured to include the display/alarm module 108 of circuit 60. For example, referring to FIG. 8, a cannula 620 is shown configured in a similar manner to cannula 520. Cannula 620 includes a cable 660 and a connector 650, cable 660 includes a plurality of conducting wires (including wires 632, 631) for electrically coupling the components of cannula 620 to ESU 50. Connector 650 is configured to be received by receptacle 62 of ESU 50. Cannula 620 further includes a module 660, which includes at least first and second indicator lights 662, 664 and at least one alarm or speaker 666. Module 660 is coupled via one or more conductors in cable 660 to processor 107 of circuit 60 and, in some embodiments, to other components in ESU 50. Processor 107 is configured to control each of the components in module 660. When processor 107 determines that distal end 622 is disposed in fat tissue, indicator light 662 may be turned on (e.g., a green light). When processor 107 determines that distal end 622 is disposed in muscle tissue, indicator light 664 may be turned on (e.g., a red light) and speaker 666 may be triggered to output an alarm sound to indicate to a user to discontinue a grafting procedure and/or to pull out end shaft 627 from patient tissue. It is to be appreciated that in this embodiment, circuit 60 may be modified to remove module 108.

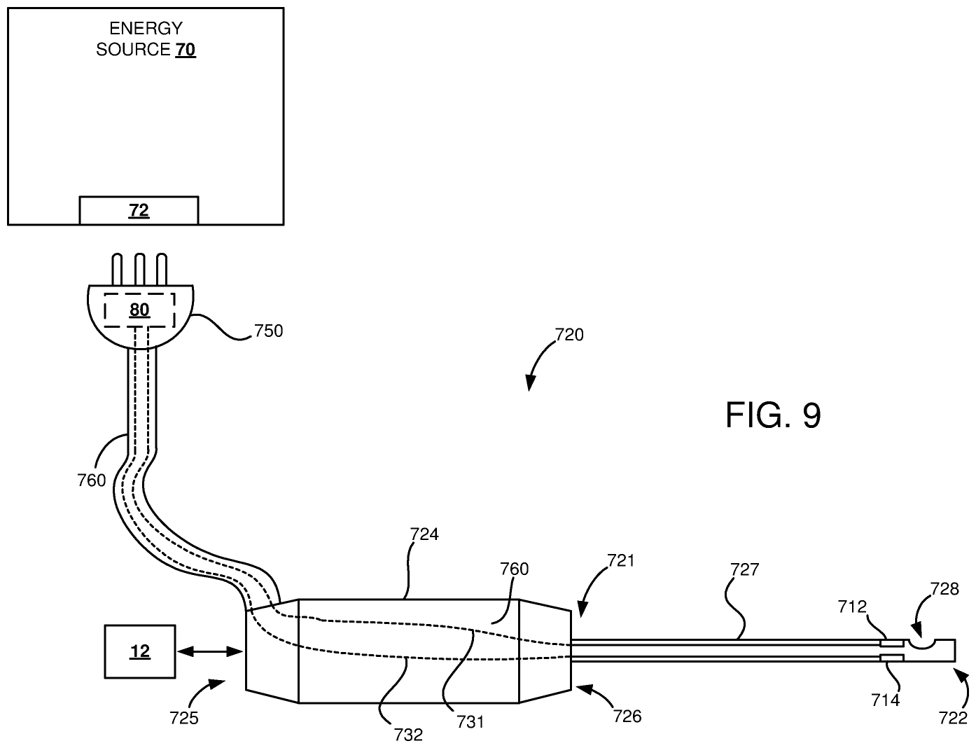

It is to be appreciated that any of the cannulas described above may be configured to include circuit 60. For example, referring to FIG. 9, a cannula 720 is shown configured in a similar manner to cannula 520 in accordance with the present disclosure. Cannula 720 includes a connector 750, which includes a circuit 80 that is configured with the same components as circuit 60. Connector 750 is configured to be received by a receptacle 72 of an energy source 70 to couple cannula 720 to energy source 70. Energy source 70 may be an ESU, such as ESU 50, or may be any other type of energy source. Source 70 is configured to provide energy to circuit 80 and to electrodes 712, 714, via wires 731, 732. Cannula 720 includes a cable 760 for coupling connector 750 to cannula 720, where wires 731, 732 are partially disposed in cable 760. In this embodiment, circuit 80 functions in the same manner as circuit 60 to enable cannula 720 to monitor the impendence of tissue proximate to electrodes 712, 714 and determine whether distal end 722 is disposed in fat tissue or muscle tissue. Although not shown, circuit 80 may be disposed in the base 724 of the cannula 720. Additionally, circuit 80 may be disposed in energy source 70 to create a stand-alone system, obviating the need for an electrosurgical generator.

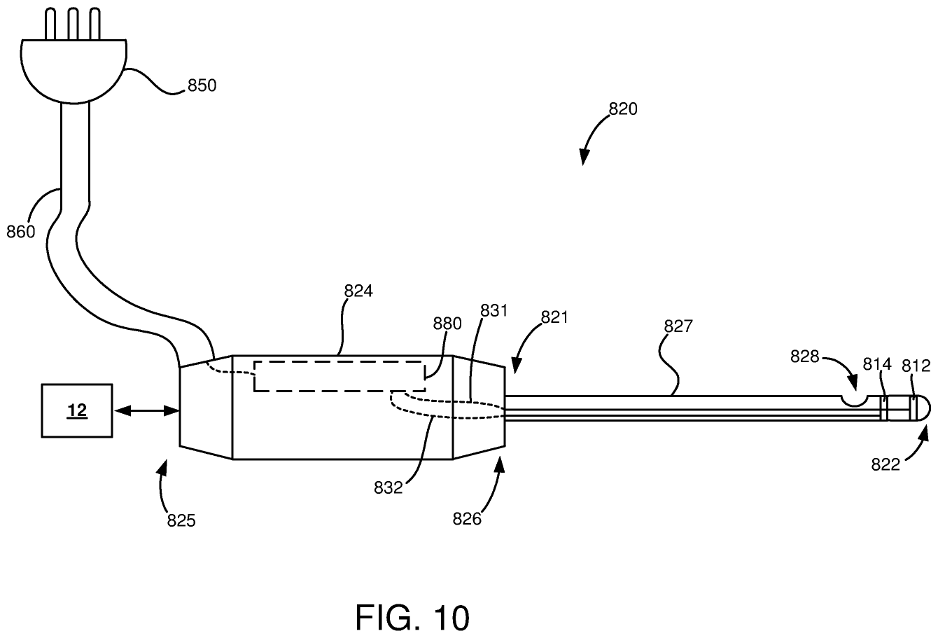

It is to be appreciated that in another embodiment of the present disclosure, acoustic impedance, rather than electrical impedance may be used to determine if the distal end of a cannula is disposed in fat tissue or muscle tissue during a fat grafting procedure. For example, referring to FIG. 10, a cannula 820 is shown including acoustic transducers 812, 814, each disposed a predetermined distance apart from each other along shaft 827 proximately to a distal end 822. One of the transducers, e.g., 812, may operate or be configured as an acoustic emitter configured to emit a sound wave having a predetermined frequency and amplitude or intensity responsive to control signals received from at least one processor. The other of the transducers, e.g. 814, may operate or be configured as an acoustic receiver configured to receive sound wave and convert the sound waves to electrical signals indicative of the properties (i.e., frequency and amplitude or intensity) of the sound wave. The electrical signals converted from the sound waves may be provided to the at least one processor.

In one embodiment, cannula 820 includes a circuit 880 including the at least one processor and other components for operating transducers 812, 814 (e.g., analog to digital converters, amplifiers, etc.) Circuit 880 is coupled to transducer 812 via conductive wire 831 and to transducer 814 via conductive wire 832. Cannula 820 includes a connector 850 and cable 860, where cable 860 includes one or more conductive wires. Circuit 880 is coupled to at least one conductive wire in cable 860. Connector 850 is configured to be received by an energy source (e.g., ESU 50, energy source 70, etc.) to provide power to circuit 880 and to transducers 812, 814.

In one embodiment, when distal end 822 of shaft 827 is inserted into a subcutaneous tissue plane, the processor in circuit 880 is configured to control emitter 812 to emit one or more sound waves having a predetermined frequency and predetermined amplitude or intensity. The sound waves are received by receiver 814, converted into electrical signals and provided to the processor of circuit 880 for processing. As the sound waves travel through different materials (e.g., blood, fat, muscle, etc.) in the tissue plane that distal end 822 is disposed in, the sound wave emitted by emitter 812 will be attenuated. If the sound waves travel through muscle tissue at any point during their propagation, the sound waves will be attenuated differently (i.e., the intensity or amplitude of the sound waves) than if the sound waves travel solely through fat tissue. Knowing the distance between the emitter 812 and receiver 814, the intensity of emitted sound waves, and the intensity of the received sound waves, the processor in circuit 880 is configured to determine if the sound waves emitted by emitter 812 have travelled through muscle tissue during their propagation. For example, consider a sound wave that has the amplitude A₀ at a specific point, i.e., the point where the sound wave leaves the emitter. After travelling a distance z from that point, i.e., the receiver is located distance z from emitter, the sound wave's amplitude A(z) will be:

A(z)=A₀e^{-αz} (5)

Where α is the frequency-dependent amplitude attenuation coefficient in neper per meter (Np/m). Attenuation coefficients of biological tissues varies with frequency and are usually reported in dB/(cm×MHz). The conversion between Np and dB is: 1 Np≈8.686 dB. Example values for the attenuation coefficient (in dB/cm at 1 MHz) are 0.61 for fat and 0.7-1.4 for muscle. As described above, knowing the distance z between the emitter 812 and receiver 814, the amplitude of the emitted sound waves A₀, and the amplitude of the received sound waves A(z), the processor in circuit 880 is configured to determine whether the sound wave past through fat tissue or muscle tissue.

In some embodiments, processor 107 executes one or more mathematical expressions designed and calibrated for use with transducers 812, 814 to different between different soft tissue types, such as fat and muscle. If the processor in circuit 880 determines that the sound waves emitted by emitter 812 and received by received 814 have travelled through muscle tissue based on the attenuation, the processor is configured to alert the user (e.g., via one or more indicator lights and/or an alarm sound).

In another embodiment, rather than using the attenuation of the waves traveling between transducers 812, 814, the processor in circuit 880 is configured to monitor the time-of-flight of the sound waves (i.e., the time it takes a sound wave to travel from emitter 812 to receiver 814). Using the time-of-flight and the known distance between transducers 812, 814, the processor in circuit 880 is configured to determine the speed of the sound waves, using the following equation:

c=z/t (6)

Where c is the speed of the sound wave in a material in meters per second, z is the distance between transducers 812, 814 in meters, and t is the time of flight in seconds. Exemplary values for the speed of sound in m/s are 1450-1460 in fat and 1550-1600 for muscle. Based, the speed of the sound waves, the processor is configured to determine if the sound waves traveled through muscle tissue or solely through fat tissue, since the speed of the sound waves is a function of the material density that the sound waves travel through. If the processor in circuit 880 determines that the sound waves emitted by emitter 812 and received by received 814 have travelled through muscle tissue based on the speed of the sound waves, e.g., the speed of sound wave is in the range of 1550-1600 m/s, the processor is configured to alert the user (e.g., via one or more indicator lights and/or an alarm sound) that the distal end of the probe is disposed in muscle tissue.

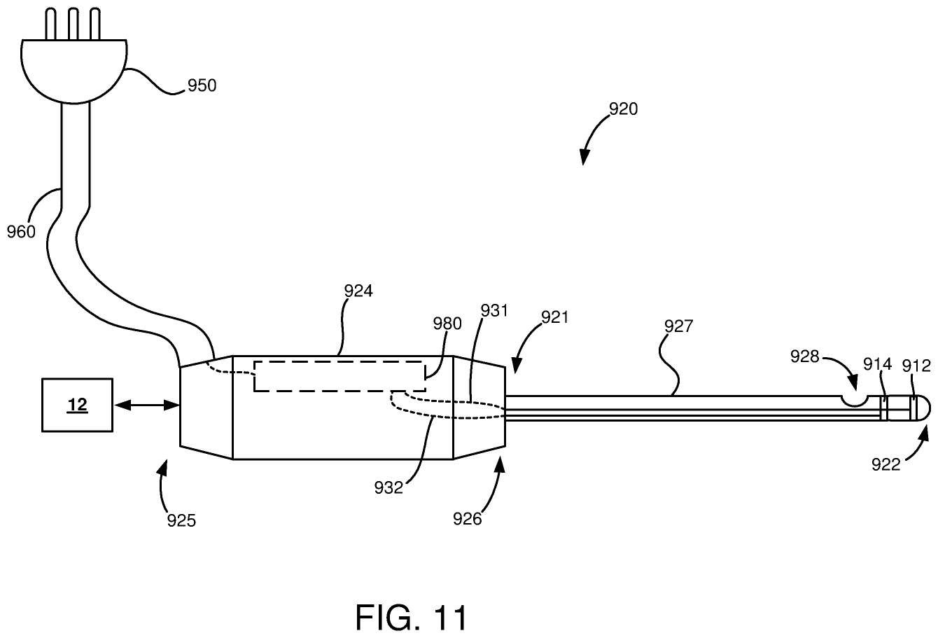

It is to be appreciated that in another embodiment of the present disclosure, the propagation of heat energy and the heat capacity of the different soft tissue types may be used to determine if the distal end of a cannula is disposed in fat tissue or muscle tissue during a fat grafting procedure. For example, referring to FIG. 11, a cannula 920 is shown including a heating element 912 and a temperature sensor 914, each disposed on distal portion of shaft 927. Element 912 and sensor 914 are coupled via wires 931, 932, respectively, to a circuit 980. Circuit 980 is coupled via cable 960 and connector 950 to an energy source (such as ESU 50) for receiving power. Circuit 980 includes a circuit 980 including at least one processor and other components for operating element 912 and sensor 914 (e.g., analog to digital converters, amplifiers, etc.)

The heat capacity of soft tissue types are different from each other, i.e., if the same amount of energy is applied to the same mass of tissues, but with different heat capacity, the temperature increase of each mass will be different. For example, fat has a much lower heat capacity than blood and muscle. The processor of controller 980 uses the differences in heat capacity between fat and muscle to differentiate between fat and muscle when end 922 is disposed in a subcutaneous tissue plane based on readings from sensor 914. The following equation may be used to calculate the temperature increase of tissues with different heat capacities:

$$Δ T = \frac{Q}{C \times m} \qquad ( 7 )$$

where ΔT is the temperature increase in Kelvin (K), Q is the applied heat energy in Joules (J), m is the mass in kilograms (kg) and C is the heat capacity in J/(K×kg). Exemplary values for heat capacity for soft tissues varies with an average value of 2348 J/(K×kg) for fat and 3421 J/(K×kg) for muscle. When distal end 922 of shaft 927 is disposed in a subcutaneous tissue plane during a fat grafting procedure, the processor of circuit 980 is configured to cause heating element 912 to send a heat pulse having a predetermined amount of energy through the tissue adjacent to end 922 of shaft 927. Sensor 914 is configured to provide one or more temperature readings of the tissue disposed adjacent to end 922 before and after element 912 outputs the heat pulse. The temperature change measured by sensor 914 of the tissue adjacent to end 922 before and after the heat pulse is outputted is determined by the processor in circuit 980 and used to determine whether end 922 is disposed in fat tissue or muscle tissue. For example, if the temperature increase of the tissue adjacent to end 922 is within a first range, the processor may determine that end 922 is disposed in fat and, if the temperature increase of the tissue adjacent to end 922 is within a second range, the processor may determine that end 922 is disposed in muscle. In addition to the equation above to determine the heat capacity ranges, it is to be appreciated that the first and second ranges may be determined by experimentation and the ranges may vary with cannula size/geometry. If the processor in circuit 980 determines that the distal end 922 of shaft 927 is disposed in muscle tissue, the processor is configured to alert the user (e.g., via one or more indicator lights and/or an alarm sound).

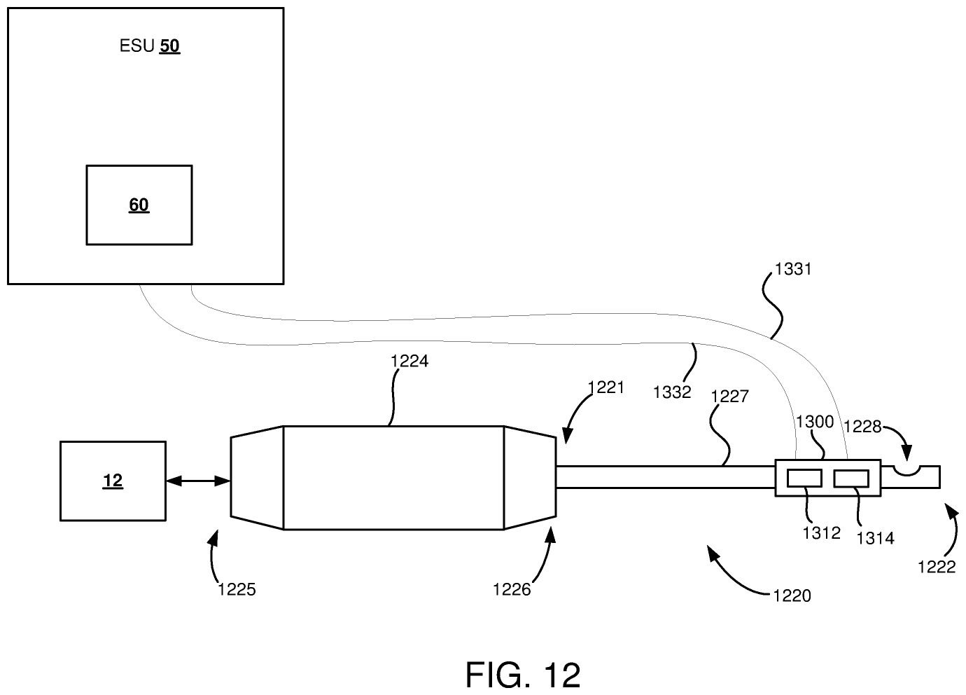

In another embodiment of the present disclosure, existing fat grafting cannulas (e.g., without muscle/fat tissue discerning capabilities) may be retrofitted with muscle/fat tissue discerning capabilities using a connector including one or more electrodes in accordance with an embodiment of the present disclosure. For example, referring to FIG. 12, a fat grafting cannula 1220 that does not include muscle/fat tissue discerning capabilities is shown. Cannula 1220 includes a base or handle 1224 with ends 1225, 1226, a shaft 1227 with ends 1221, 1222 and aperture(s) 1228). In one embodiment, the present disclosure provides a connector 1300, e.g., a sheath, that is configured to be coupled to a portion of shaft 1227 (e.g., a distal portion) via a coupling mechanism (e.g., clamps, adhesives, and/or securing members, etc.). It is to be appreciated that connector 1300 may be removably coupled to the shaft 1227, i.e., the connector 1300 may be disposed of after one use while the probe/cannula may be reused with a new connector 1330. The connector 1300 includes electrodes 1312, 1314, which may be configured to sense at least one property (e.g., electrical property, acoustic property, heat property) of the tissue adjacent to end 1222 of cannula 1220 in the manner described above with respect to other embodiments of the present disclosure. Each electrode 1312, 1314 is coupled to circuit 60 of ESU 50 via a corresponding conductive wire 1331, 1332 to provide signals indicative of the sensed at least one property to processor 107 in circuit 60 to determine whether tip 1222 of cannula 1220 is disposed in muscle tissue or fat tissue in the manner described above. In this way, connector 1300 enables a cannula 1200 to be retrofitted with muscle/fat tissue discerning capabilities described herein.

It is to be appreciated that, although connector 1300 is shown including two electrodes 1312, 1314, making connector 1300 suitable for use in a bipolar arrangement, in other embodiments of the present disclosure, connector 1300 may include a single electrode 1312 (e.g., an active electrode) and a return electrode or pad may be employed for use in a monopolar arrangement (e.g., as described in association with FIG. 3 above). In another embodiment, electrodes 1312, 1314 may be individually placed on shaft 1127 without connector 1300. In this embodiment, electrodes 1312, 1314 may include ring electrodes which can be slid over the distal end 1222 of shaft 1227 and positioned as desired by a user. The ring electrodes may be set in place on the shaft 1227 by any known means including, but not limited to, adhesives. Each ring electrode may then be coupled to circuit 60 via wires 1331, 1132. Additionally, wires 1331, 1332 may be coupled to a second connector, i.e., a connector similar to connector 550, 650, 705, that is configured to mate with an appropriate port, e.g., monopolar port 1073, bipolar port 1075, etc., of ESU 50. It is further to be appreciated that connector 1300 and/or electrodes 1312, 1314 (when not used with connector 1300) may be coupled to other power sources as described above in lieu of ESU 50, for example, when a power source and circuit 60 are a standalone device not requiring ESU 50.

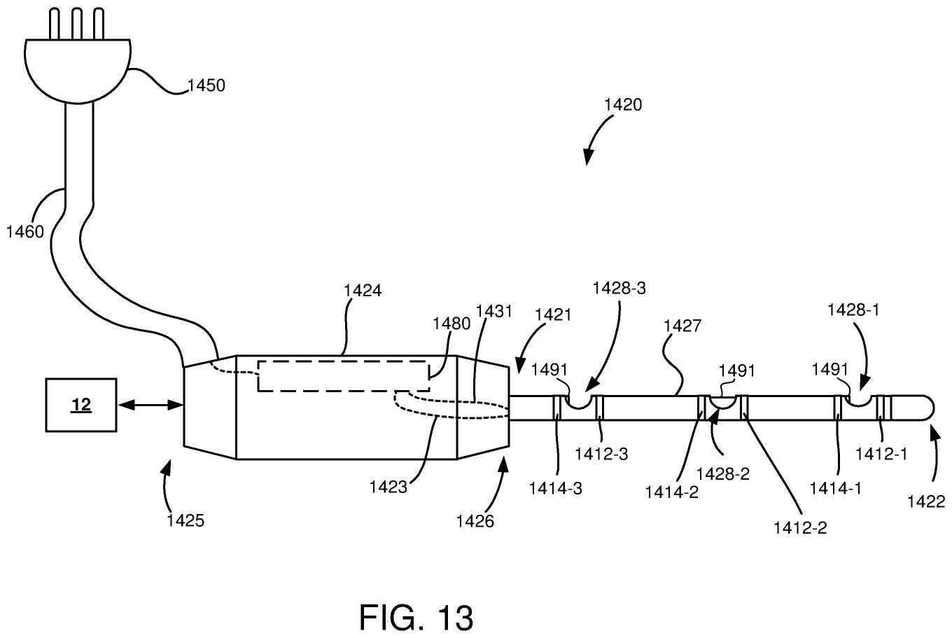

It is to be appreciated that in another embodiment of the present disclosure, the cannula or probe may include more than one aperture for inserting processed fat into a layer of fat tissue of a patient. For example, referring to FIG. 13, a cannula 1420 is shown including three apertures 1428-1, 1428-2 and 1428-3. It is to be appreciated that although three apertures are shown, the present disclosure contemplates at least one or more apertures disposed on various locations of the shaft 1427. Each aperture 1428 is associated with a first sensor 1412, e.g., sensors 1412-1, 1412-2, 1412-3, and a second sensor 1414, e.g., 1414-1, 1414-2, 1414-3. In one embodiment, each of the sensors 1412, 1414 are disposed on opposite sides of a corresponding aperture 1428. The first sensor 1412 and second sensor 1414 may then be employed by circuit 1480 to determine if the corresponding aperture 1428 is disposed in fat tissue or muscle tissue.

In one embodiment, cannula 1420 includes a circuit 1480 including the at least one processor and other components for operating sensors 1412, 1414 (e.g., analog to digital converters, amplifiers, etc.) Circuit 1480 is coupled to sensor 1412 via at least one conductive wire 1431 and to sensor 1414 via at least one conductive wire 1432. It is to be appreciated that each sensor may be individually coupled to circuit 1480 via a single or multi-conductor wire. As described above in relation to the other embodiments, circuit 1480 may be disposed in the base 1424 of cannula 1420, ESU 50 or in a stand-alone device.

Sensors 1412, 1414 may include any sensor that can enable the circuit 1480 to discern between fat tissue and muscle tissue and may include, but are not limited to, any sensor described above including electrodes, acoustic transmitters, acoustic receivers, heating elements, temperature sensors, etc. The circuit 1480 may discern or determine if the aperture 1428 is disposed in fat tissue or muscle tissue by at least any of the methods described above including by electrical impedance, acoustic impedance, heat capacity, etc.

Cannula 1420 includes a connector 1450 and cable 1460, where cable 1460 includes one or more conductive wires. Circuit 1480 is coupled to at least one conductive wire in cable 1460. Connector 1450 is configured to be received by an energy source (e.g., ESU 50, energy source 70, etc.) to provide power to circuit 1480 and to sensors 1412, 1414.

In one embodiment, each aperture may include a door 1491 and actuator (not shown) for opening and closing the door 1491. Each door 1491 may be individually controllable by circuit 1480. The circuit 1480 may close a respective door 1491 when the sensors associated with a particular aperture are used to determine that the particular aperture is disposed in muscle tissue. If the circuit 1480 determines a particular aperture is disposed in fat tissue, the circuit 1480 may open the corresponding door 1491 to allow processed fat to flow through that aperture. For example, referring to FIG. 13, circuit 1480 may determine that the aperture 1428-1 and aperture 1428-3 are disposed in fat tissue based on the properties sensed by sensors 1412-1, 1414-1, 1412-3, 1414-3 respectively. Additionally, circuit 1480 may determine that the aperture 1428-2 is disposed in muscle tissue based on the properties sensed by sensors 1412-2, 1414-2. In the example shown in FIG. 13, circuit 1480 may close door 1491 to prevent processed fat to flow from aperture 1428-2 into muscle tissue, while the doors associated with apertures 1428-1 and 1428-3 remain open to allow processed fat to follow therefrom.

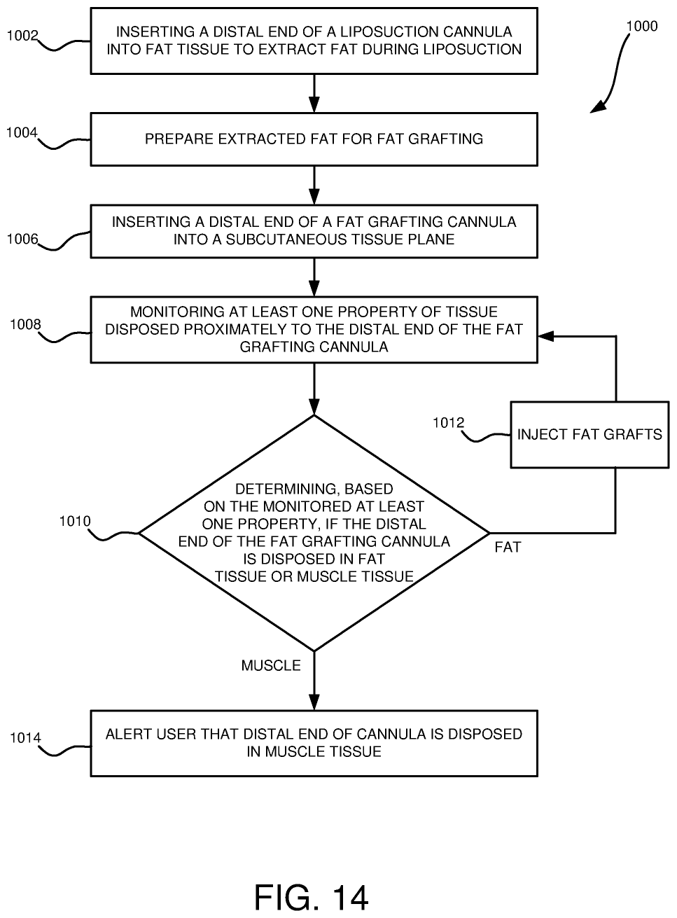

Referring FIG. 14, a method 1000 for using muscle and/or fat discerning cannulas to perform fat grafting is shown in accordance with an embodiment of the present disclosure. It is to be appreciated that the method 1000 of FIG. 14 may be performed using any of the fat grafting cannulas described above. In step 1002, the distal end of a liposuction cannula is disposed into an adipose of fat tissue plane to extract fat during liposuction. In step 1004, the extracted fat is prepared or processed for use in fat grafting. In step 1006, the distal end of a fat cannula (e.g. any of the cannulas described above) is inserted into a subcutaneous tissue plane to inject the processed fat. In step 1010, based on at least one property (e.g., electrical impedance, acoustic impedance, heat capacity, etc.) associated with the tissue adjacent to the distal end of the cannula, a processor (e.g., in a circuit, such as circuit 60, 80, etc.) is configured to determine if the distal end of the fat grafting cannula is disposed in fat tissue or in muscle tissue.

If the processor determines that the distal end of the fat grafting cannula is disposed in fat tissue, the user may be alerted (e.g., via indicator light(s) and/or of the cannula and/or generator the cannula is coupled to, as described above) and the user of the fat grafting cannula may proceed to inject the fat grafts, in step 1012. Thereafter, steps 1008 and 1010 will be repeatedly performed until fat injection has finished to monitor the at least one property associated with the tissue to ensure the distal end of the fat grafting cannula is not disposed in muscle tissue. Alternatively, if in step 1010 it is determined by the processor that the distal end of the fat grafting cannula is disposed in muscle tissue, the user is alerted (e.g., via indicator light(s) and/or an alarm sound produced from a circuit of the fat grafting cannula and/or device the cannula is coupled to, as described above) that the distal end of the fat grafting cannula is disposed in muscle, in step 1014. In this way, the user does not begin or stops the continuation of any fat injection into the muscle tissue the distal end is disposed in. In some embodiments, in step 1014, the processor sends one or more signals to a pressure control device coupled to the fat grafting cannula to stop or block processed fat from flowing through the cannula and being injected into the muscle tissue.

It is to be appreciated that method 1000 may be performed using ESU 50 (e.g., including circuit 60) described above during a body contouring procedure.

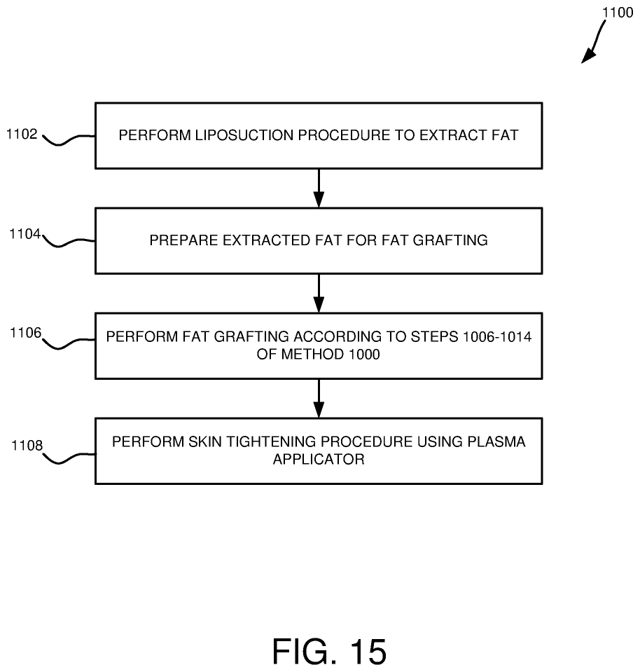

For example, referring to FIG. 15 a method 1100 for performing body contouring procedures using a fat/muscle discerning cannula (e.g., any of the cannulas described above for use with ESU 50), ESU 50, and a plasma applicator is shown in accordance with an embodiment of the present disclosure. In step 1102, the distal end of a liposuction cannula (i.e., any of the cannulas described above) is disposed into an adipose or fat tissue plane to extract fat during liposuction. In step 1104, the extracted fat is prepared or processed for use in fat grafting. In step 1106, the distal end of a fat grafting cannula coupled to ESU 50 and configured to discern between fat and muscle (i.e., any of the cannulas described above) is inserted into a subcutaneous tissue plane and used to perform fat grafting according to steps 1006-1014 in method 1000. In some embodiments, circuit 60 of ESU 50 may be used to determine whether the distal end of the cannula is disposed in fat or muscle tissue during the fat grafting. Alternatively, a circuit in the cannula may be used to determine whether the distal end of the cannula is disposed in fat or muscle tissue. In step 1108, after the fat grafting is completed, a plasma applicator is coupled to ESU 50 for receiving electrosurgical energy therefrom, and the plasma applicator is used to perform a skin tightening procedure on the skin surface above the tissue area where the fat was injected or grafted to in step 1106. It is to be appreciated that steps 1102-1108 may be performed during a single procedure, or alternatively, certain steps may be performed at different times or as a separate procedure. For example, steps 1102 and 1104 may be performed to extract fat and prepare or process the fat for grafting at a later time. During a subsequent procedure, the processed fat may be grafted into the patient, as described in step 1106. Furthermore, a skin tightening procedure, as described in relation to step 1108, may be performed as a subsequent procedure after a fat grafting procedure was conducted.

It is to be appreciated that while the embodiments above describe devices and systems for use in fat grafting, in other embodiments of the present disclosure, the devices and systems above may be implemented for use with any cannula or devices which is placed blindly by the user into subcutaneous tissue. For example, the devices and systems above could be implemented for liposuction cannulas and infiltration cannulas (e.g., used for placing tumescent anesthesia) to give the user an indication that they are in the proper location.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. A fat grafting probe comprising:
a base having a proximal end and a distal end, the base including a first fluid channel extending between the proximal and distal ends, the proximal end including an opening configured to receive an output of a pressure control device;
a shaft including a proximal end coupled to the distal end of the base and a distal end including at least one aperture, the shaft includes a hollow interior operating as a second fluid channel which is in fluid communication with the first fluid channel;
at least two electrodes associated with the shaft and coupled to an impedance detection circuit; and
the impedance detection circuit that determines the impedance between the at least two electrodes and generates an indication whether the distal end of the shaft is in fat tissue or muscle tissue.

2. The probe of claim 1, wherein the impedance detection circuit includes:
a transformer having primary windings and secondary windings, the at least two electrodes being coupled to the secondary windings;
a voltage controlled alternating current source coupled to one leg of the primary windings; and
at least one processor coupled to the one leg of the primary windings to sense voltage on the one leg and determine the impedance between the at least two electrodes based on the sensed voltage.

3. The probe of claim 1, wherein the impedance detection circuit further includes an interface module that provides the indication whether the distal end of the shaft is in fat tissue or muscle tissue.

4. The probe of claim 3, wherein the interface module is disposed on the base.

5. The probe of claim 2, wherein the impedance detection circuit further includes a low pass filter disposed between the second windings and the at least two electrodes to suppress radio frequency noise.

6. The probe of claim 1, wherein if the impedance detection circuit determines the impedance is below a first predetermined setpoint, the distal end of the shaft is disposed in muscle tissue.

7. The probe of claim 6, wherein if the impedance detection circuit determines the impedance is above a second predetermined setpoint, the distal end of the shaft is disposed in fat tissue.

8. The probe of claim 2, further comprising a communication module coupled to the at least one processor, the communication module communicates the indication to at least one other device.

9. The probe of claim 1, wherein a first electrode of the at least two electrodes is disposed on the distal end of the shaft and a second electrode is a return pad electrode.

10. The probe of claim 9, wherein the shaft is configured from a conductive material with an insulative sheath covering at least a portion of the shaft, an exposed portion of the shaft forming the first electrode.

11. The probe of claim 1, wherein the shaft is configured from a conductive material with an insulative sheath covering at least a portion of the shaft, an exposed portion of the shaft forming a first electrode and a second electrode disposed on the sheath.

12. The probe of claim 1, wherein the at least two electrodes are disposed at selected positions on the shaft, a first electrode being disposed a predetermined distance from a second electrode.

13. The probe of claim 1, further comprising a connector for coupling conductive wire of the at least two electrodes to a power source, the connector including at least one memory configured to store parameters associated with the probe.

14. The probe of claim 1, further comprising a connector for coupling conductive wire of the at least two electrodes to a power source, wherein at least a portion of the impedance detection circuit is disposed in the connector.

15. The probe of claim 1, wherein the at least two electrodes are disposed on a connector that is removably coupled to the shaft.

16. A fat grafting system comprising:
an electrosurgical generator configured for providing power;
a probe coupled to the electrosurgical generator including:
a base having a proximal end and a distal end, the base including a first fluid channel extending between the proximal and distal ends, the proximal end including an opening configured to receive an output of a pressure control device;
a shaft including a proximal end coupled to the distal end of the base and a distal end including at least one aperture, the shaft includes a hollow interior operating as a second fluid channel which is in fluid communication with the first fluid channel; and
at least two electrodes associated with the shaft and coupled to an impedance detection circuit; and
the impedance detection circuit that determines the impedance between the at least two electrodes and generates an indication whether the distal end of the shaft is in fat tissue or muscle tissue.

17. The system of claim 16, wherein the impedance detection circuit is disposed in the generator.

18. The system of claim 16, wherein the pressure control device provides processed fat to a layer of fat tissue of a patient via the first fluid channel, the second fluid channel and the at least one aperture.

19. The system of claim 18, wherein the pressure control device is at least one of a syringe and/or a pump.

20. The system of claim 17, wherein the impedance detection circuit includes:
a transformer having primary windings and secondary windings, the at least two electrodes being coupled to the secondary windings;
a voltage controlled alternating current source coupled to one leg of the primary windings; and
at least one processor coupled to the one leg of the primary windings to sense voltage on the one leg and determine the impedance between the at least two electrodes based on the sensed voltage.

21. The system of claim 20, wherein the impedance detection circuit further includes an interface module that provides the indication whether the distal end of the shaft is in fat tissue or muscle tissue.

22. The system of claim 21, further comprising a display module coupled to the interface module configured to display the indication, the display module disposed on a surface of a housing of the electrosurgical generator.

23. The system of claim 20, wherein the electrosurgical generator is further configured to be coupled to a plasma generator and provide an electrosurgical radio frequency signal to the plasma generator.

24. The system of claim 23, wherein a frequency of an output of the voltage controlled alternating current source is selected to be different than a frequency of the electrosurgical radio frequency signal.

25. The system of claim 18, wherein the impedance detection circuit further comprising a communication module coupled to the at least one processor, the communication module communicates a control signal to the pressure control device when the at least one processor determines that the distal end of the shaft is in muscle tissue.

26. The system of claim 16, wherein the probe further comprising a connector for coupling conductive wire of the at least two electrodes to the electrosurgical generator, the connector including at least one memory configured to store parameters associated with the probe and transmit the parameters to at least one processor of the electrosurgical generator.

27. A fat grafting probe comprising:
a base having a proximal end and a distal end, the base including a first fluid channel extending between the proximal and distal ends, the proximal end including an opening configured to receive an output of a pressure control device;
a shaft including a proximal end coupled to the distal end of the base and a distal end including at least one aperture, the shaft includes a hollow interior operating as a second fluid channel which is in fluid communication with the first fluid channel;
at least two sensors associated with the shaft and coupled to a detection circuit; and
the detection circuit that determines whether the distal end of the shaft is in fat tissue or muscle tissue based on sensed parameters of the at least two sensors.

28. The probe of claim 27, wherein the at least two sensors include an acoustic emitter and an acoustic receiver, the acoustic emitter disposed a predetermined distance from the acoustic receiver,
the detection circuit including at least one processor configured to determine attenuation of a signal emitted by the acoustic emitter and determines whether the distal end of the shaft is in fat tissue or muscle tissue based on the attenuated signal.

29. The probe of claim 28, wherein the signal emitted by the acoustic emitter has at least one of a predetermined frequency and/or a predetermined amplitude.

30. The probe of claim 27, wherein the at least two sensors include an acoustic emitter and an acoustic receiver, the acoustic emitter disposed a predetermined distance from the acoustic receiver,
the detection circuit including at least one processor configured to determine a time of flight of a signal emitted by the acoustic emitter to the acoustic receiver and determines whether the distal end of the shaft is in fat tissue or muscle tissue based on the speed of the signal.

31. The probe of claim 27, wherein the at least two sensors include a heating element and a temperature sensor, the heating element disposed a predetermined distance from the heat sensor,
the detection circuit including at least one processor configured to determine heat capacity of tissue between the heating element and heat sensor and determines whether the distal end of the shaft is in fat tissue or muscle tissue based on the determined heat capacity.

32. The probe of claim 31, wherein the at least one processor determines the heat capacity by measuring a temperature difference sensed by the temperature sensor before and after a predetermined heat pulse is emitted by the heating element.

33. The probe of claim 28, wherein the at least two sensors are disposed on a connector that is removably coupled to the shaft.

34. A method for performing a medical procedure comprising:
inserting a distal end of a fat grafting cannula into a subcutaneous tissue plane;
monitoring at least one property of tissue disposed proximately to the distal end of the fat grafting cannula,
determining, based on the monitored at least one property, if the distal end of the fat grafting cannula is disposed on in fat tissue or muscle tissue; and
generating an indication of whether the distal end is in fat tissue or muscle tissue.

35. The method of claim 34, further comprising, if the distal end of the fat grafting cannula is disposed in fat tissue, generating an alert to proceed to inject processed fat into the fat tissue.

36. The method of claim 34, further comprising, if the distal end of the fat grafting cannula is disposed in fat tissue, transmitting a signal to a processed fat pressure controlling device to proceed to inject processed fat into the fat tissue via the fat grafting cannula.

37. The method of claim 36, further comprising, if the distal end of the fat grafting cannula is disposed in muscle tissue, transmitting a signal to the processed fat pressure controlling device to stop providing processed fat to the fat grafting cannula.

38. The method of claim 34, further comprising, if the distal end of the fat grafting cannula is disposed in muscle tissue, generating an alert that the distal end of the fat grafting cannula is disposed in muscle tissue.

39. The method of claim 34, wherein the at least one property includes at least one of electrical impedance, acoustic impedance and/or heat capacity.

40. The method of claim 35, further comprising extracting fat from a fat layer of a patient and processing the fat for grafting into the patient.

41. The method of claim 40, further comprising performing a tissue tightening procedure after injecting the processed fat into the fat tissue.
